(19) United States
(12) Patent Application Publication
(10) Pub. No.: US 2018/0134683 A1
(43) Pub. Date: May 17, 2018

(54) HETEROCYLCIC COMPOUNDS AS IDH2 INHIBITORS

(71) Applicant: TELIGENE LTD., Suzhou (CN)

(72) Inventors: Xiaoyang Xia, Thousand Oaks, CA (US); Dawei Zhang, Thousand Oaks, CA (US)

(73) Assignee: TELIGENE LTD., Suzhou (CN)

(21) Appl. No.: 15/571,844

(22) PCT Filed: May 6, 2016
(86) PCT No.: PCT/CN2016/081307

§ 371 Date: Nov. 5, 2017

Related U.S. Application Data
(60) Provisional application No. 62/179,398, filed on May 7, 2015.

(51) Int. Cl. C07D 401/14; C07D 401/04; C07D 413/14; A61P 35/00

(52) U.S. Cl. CPC C07D 401/14; A61P 35/00; C07D 413/14; C07D 401/04

(57) ABSTRACT

The present invention is directed to novel heterocyclic compounds, their derivatives, pharmaceutically acceptable salts, solvates and hydrates thereof. The compounds and compositions of the present invention have IDHs inhibitory activities and are useful for the treatment of Isocitrate dehydrogenases mediated diseases and conditions. Novel heterocyclic compounds disclosed herein include pyrimidines and triazines.

### FIELD OF THE INVENTION

The present invention is directed to inhibitors of enzymes and pharmaceutically acceptable salts, solvates, hydrates, prodrugs and metabolites thereof, the preparation method thereof, and the use of such compounds to treat Isocitrate dehydrogenases mediated diseases and conditions such as cancer.

### BACKGROUND OF THE INVENTION

Isocitrate dehydrogenases (IDHs) catalyze the oxidative decarboxylation of isocitrate to 2-oxoglutarate (i.e., a-ketoglutarate). These enzymes belong to two distinct subclasses, one of which utilizes NAD (+) as the electron acceptor and the other NADP (+). Five isocitrate dehydrogenases have been reported: three NAD (+)-dependent isocitrate dehydrogenases, which localize to the mitochondrial matrix, and two NADP (+)-dependent isocitrate dehydrogenases, one of which is mitochondrial and the other predominantly cytosolic. Each NADP (+) dependent isozyme is a homodimer

Human IDH2 gene encodes a protein of 452 amino acids. The nucleotide and amino acid sequences for IDH2 can be found as GenBank entries NM_002168.2 and NP 002159.2 respectively. The nucleotide and amino acid sequence for human IDH2 are also described in, e.g., Huh et al., Submitted (NOV-1992) to the EMBLIGenBanklDDBJ databases; and The MGC Project Team, Genome Res. 14:2121-2127(2004).

It has been discovered that mutations of IDH 1/2 present in certain cancer cells result in a new ability of the enzyme to catalyze the NAPH-dependent reduction of α-ketoglutarate to R(−)-2-hydroxyglutarate (2HG). 2HG is not formed by wild-type IDH 1/2. The production of 2HG is believed to contribute to the formation and progression of cancer (Dang, L et al, Nature 2009,462:739-44). The inhibition of mutant IDH 1/2 and their alpha hydroxyl neoactivity are therefore a potential therapeutic treatment for cancer. Accordingly, there is an ongoing need for inhibitors of IDH 1/2 mutants having alpha hydroxyl neoactivity.

### SUMMARY OF THE INVENTION

In some embodiments of the present invention, there are provided compounds of Formula I:

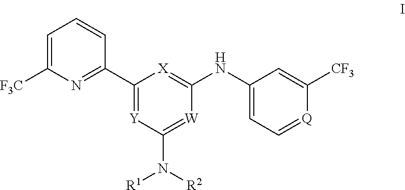

or a pharmaceutically acceptable salt, solvate or a prodrug or a stereoisomer or a tautomer or a metabolite thereof, wherein
R¹ and R² are independently —C(O)OR³, —C(O)R³, —SO₂R³, or C₁-C₆ alkyl, optionally substituted by one or more R⁴, or R¹ and R² are optionally taken together with the nitrogen they are attached to form 3-12 membered heteroalicyclic ;
R³ is C₁-C₆ alkyl, optionally substituted by one or more R⁴;
R⁴ is halogen, OH, C₁-C₆ alkoxy, NH(CO)R⁵R⁶, or NR⁵R⁶;
R⁵ and R⁶ are independently hydrogen or C₁-C₆ alkyl;
X, Y, W and Q are N or CH.

In some embodiments of the present invention, there are provided compounds of Formula II:

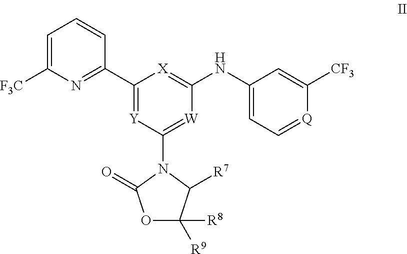

or a pharmaceutically acceptable salt, solvate or a prodrug or a stereoisomer or a tautomer or a metabolite thereof, wherein
R⁷, R⁸ and R⁹ are independently hydrogen, C₁-C₆ alkyl, optionally substituted by one or more R⁴;
X, Y, W and Q are N or CH.
The present invention further provides pharmaceutical compositions comprising a compound of Formula I or II described above and a pharmaceutically acceptable carrier.
The present invention further provides methods for regulating Isocitrate dehydrogenases comprising administrating to a mammalian subject a therapeutically effective amount of any of the compounds of Formula I or II described above.

### DETAILED DESCRIPTION OF THE INVENTION

In some embodiments of the present invention, there are provided compounds of

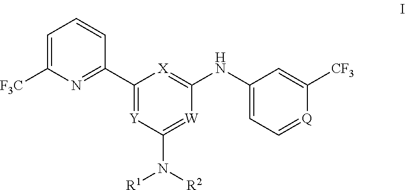

or a pharmaceutically acceptable salt, solvate or a prodrug or a stereoisomer or a tautomer or a metabolite thereof, wherein
R¹ and R² are independently —C(O)OR³, —C(O)R³, —SO₂R³, or C₁-C₆ alkyl, optionally substituted by one or more R⁴, or R¹ and R² are optionally taken together with the nitrogen they are attached to form 3-12 membered heteroalicyclic;
R³ is C₁-C₆ alkyl, optionally substituted by one or more R⁴;
R⁴ is halogen, OH, C₁-C₆ alkoxy, NH(CO)R⁵R⁶, or NR⁵R⁶;
R⁵ and R⁶ are independently hydrogen or C₁-C₆ alkyl;
X, Y, W and Q are N or CH.

In some embodiments of the present invention, there are provided compounds of Formula II:

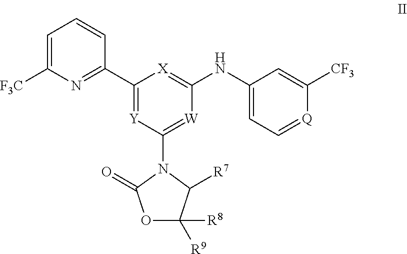

or a pharmaceutically acceptable salt, solvate or a prodrug or a stereoisomer or a tautomer or a metabolite thereof, wherein
R⁷, R⁸ and R⁹ are independently hydrogen, or C₁-C₆ alkyl, optionally substituted by one or more R⁴;
R⁴ is halogen, OH, C₁-C₆ alkoxy, NH(CO)R⁵R⁶, or NR⁵R⁶;
R⁵ and R⁶ are independently hydrogen or C₁-C₆ alkyl;
X, Y, W and Q are N or CH.
In other embodiments, the compound of Formula I or II is in the form of pharmaceutically acceptable salt. In some embodiments, the compound of Formula I or II is in the form of a solvate. In other embodiments, the compound of Formula I or II is in the form of a metabolite. In other embodiments, the compound of Formula I or II is in the form of a prodrug. In some embodiments, the compound of Formula I or II is a stereoisomer. In other embodiments, the compound of Formula I or II is a tautomer. In another embodiment, the deuterium enrichment in compounds of Formula I or II is at least about 1%.
In one embodiment, X, Y, W and Q are N.
In another embodiment, X, W and Q are N, Y is CH.

In certain embodiments, there are provided compounds without limitation selected from the group consisting of:

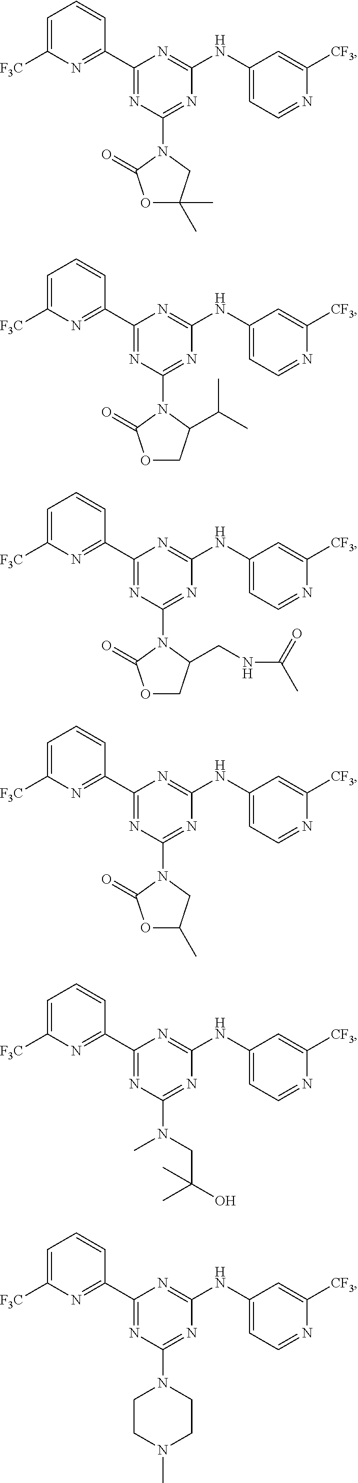 [SMILES: CC1(C)CN(c2nc(Nc3ccnc(C(F)(F)F)c3)nc(-c3cccc(C(F)(F)F)n3)n2)C(=O)O1 ; CC(C)C1COC(=O)N1c1nc(Nc2ccnc(C(F)(F)F)c2)nc(-c2cccc(C(F)(F)F)n2)n1 ; CC(=O)NCC1COC(=O)N1c1nc(Nc2ccnc(C(F)(F)F)c2)nc(-c2cccc(C(F)(F)F)n2)n1 ; CC1CN(c2nc(Nc3ccnc(C(F)(F)F)c3)nc(-c3cccc(C(F)(F)F)n3)n2)C(=O)O1 ; CN(CC(C)(C)O)c1nc(Nc2ccnc(C(F)(F)F)c2)nc(-c2cccc(C(F)(F)F)n2)n1 ; CN1CCN(c2nc(Nc3ccnc(C(F)(F)F)c3)nc(-c3cccc(C(F)(F)F)n3)n2)CC1]

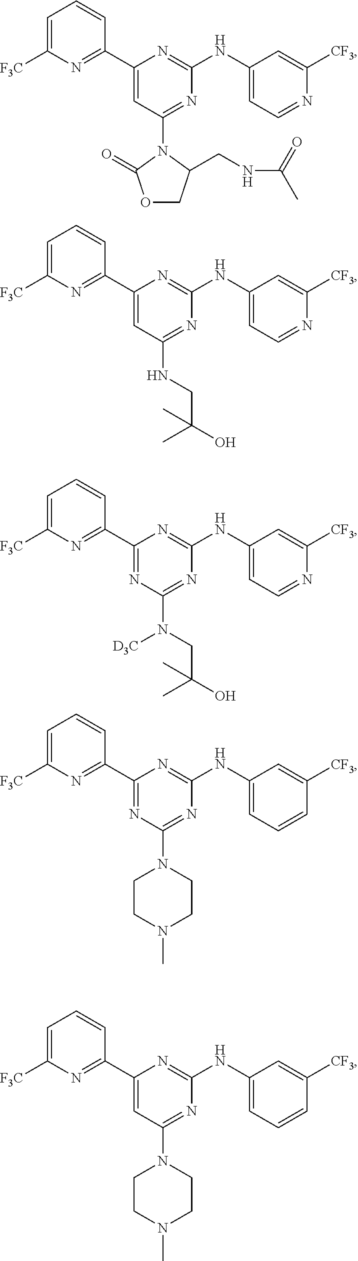 [SMILES: CC(=O)NCC1COC(=O)N1c1cc(-c2cccc(C(F)(F)F)n2)nc(Nc2ccnc(C(F)(F)F)c2)n1 ; CC(C)(O)CNc1cc(-c2cccc(C(F)(F)F)n2)nc(Nc2ccnc(C(F)(F)F)c2)n1 ; [2H]C([2H])([2H])N(CC(C)(C)O)c1nc(Nc2ccnc(C(F)(F)F)c2)nc(-c2cccc(C(F)(F)F)n2)n1 ; CN1CCN(c2nc(Nc3cccc(C(F)(F)F)c3)nc(-c3cccc(C(F)(F)F)n3)n2)CC1 ; CN1CCN(c2cc(-c3cccc(C(F)(F)F)n3)nc(Nc3cccc(C(F)(F)F)c3)n2)CC1]

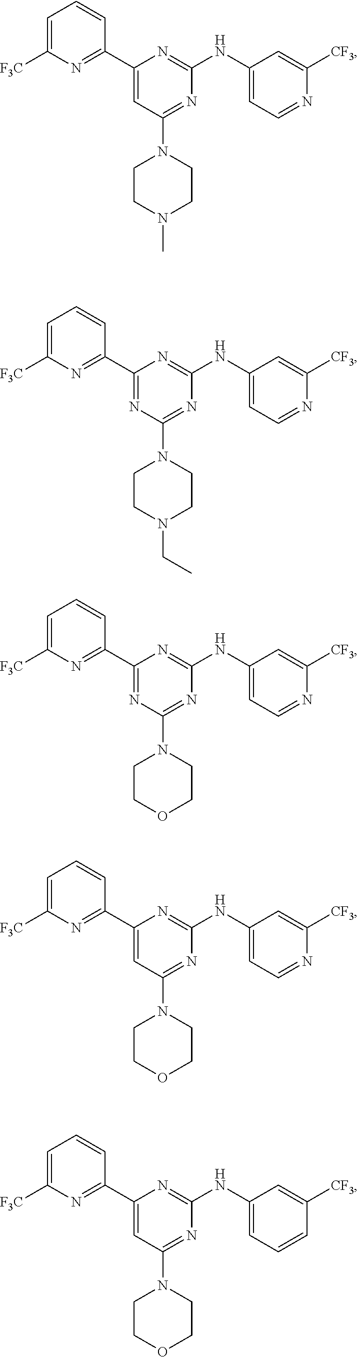 [SMILES: CN1CCN(c2cc(-c3cccc(C(F)(F)F)n3)nc(Nc3ccnc(C(F)(F)F)c3)n2)CC1 ; CCN1CCN(c2nc(Nc3ccnc(C(F)(F)F)c3)nc(-c3cccc(C(F)(F)F)n3)n2)CC1 ; FC(F)(F)c1cc(Nc2nc(-c3cccc(C(F)(F)F)n3)nc(N3CCOCC3)n2)ccn1 ; FC(F)(F)c1cc(Nc2nc(-c3cccc(C(F)(F)F)n3)cc(N3CCOCC3)n2)ccn1 ; FC(F)(F)c1cccc(Nc2nc(-c3cccc(C(F)(F)F)n3)cc(N3CCOCC3)n2)c1]

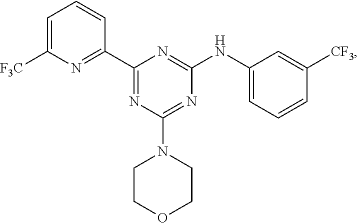 [SMILES: FC(F)(F)c1cccc(Nc2nc(-c3cccc(C(F)(F)F)n3)nc(N3CCOCC3)n2)c1]

and the like, or a pharmaceutically acceptable salt, solvate, or a prodrug, or a metabolite thereof. In some embodiments, there are provided pharmaceutical compositions comprising a compound of Formula I or II and a pharmaceutically acceptable carrier. In certain embodiments, the compositions are for the treatment of a hyper-proliferative disorder. In some embodiments, the pharmaceutical compositions further comprise an anti-neoplastic agent, an immunosuppressant, an immunostimulant, or combination thereof. In other embodiments, the pharmaceutical compositions are suitable for oral, parenteral, or intravenous administration.

In some embodiments, the present invention provides methods for regulating the Isocitrate dehydrogenases activities, said methods comprising administrating to a mammalian subject a therapeutically effective amount of a compound of Formula I or II.

In other embodiments, there are provided herein methods for treating or preventing Isocitrate dehydrogenases mediated disorder, said method comprising administrating to a mammalian subject a therapeutically effective amount of a compound of Formula I or II.

### Definitions

The term “alkyl” is intended to include straight, branched, and cyclic hydrocarbon groups, which contain only single carbon-carbon bonds and which may be unsubstituted or optionally substituted with one or more functional groups. Representative examples include methyl, ethyl, propyl, isopropyl, cyclopropyl, butyl, isobutyl, tert-butyl, cyclobutyl, pentyl, cyclopentyl, hexyl, and cyclohexyl, all of which may be optionally substituted. The preferred chain length of an alkyl group is from 1 to 6 carbon atoms. C₁-C₆ alkyl is intended to include C₁, C₂, C₃, C₄, C₅ and C₆ alkyl groups. Alkyl may be substituted or unsubstituted. Typical substituent groups include cycloalkyl, aryl, heteroaryl, heteroalicyclic, hydroxy, alkoxy, aryloxy, mercapto, alkylthio, arylthio,cyano, halo, carbonyl, thiocarbonyl, O-carbamyl, N-carbamyl, O-thiocarbamyl, N-thiocarbamyl, C-amido, N-amido, C-carboxy, O-carboxy, nitro, silyl, amino and —NR^{X}R^{Y}, wherein R^{X} and R^{Y} are independently selected from the group consisting of hydrogen, alkyl, cycloalkyl, aryl, carbonyl, acetyl, sulfonyl, trifluoromethanesulfonyl and, combined, a five- or six-member heteroalicyclic ring. Illustrative substituted alkyl group include, but are not limited to, fluoromethyl, difluoromethyl, trifluoromethyl, hydoxymethyl, methoxymethyl, 2-fluoroethyl, 2-methoxyethyl, etc.

The term “alkoxy” refers to both an —O-(alkyl) or an —O-(unsubstituted cycloalkyl) group. C₁-C₆ alkoxy is intended to include C₁-C₆ alkyl groups, wherein C₁-C₆ alkyl is defined above. Representative examples include, but are not limited to, methoxy, ethoxy, propoxy, butoxy, cyclopropyloxy, cyclobutyloxy, cyclopentyloxy, cyclohexyloxy, and the like.

“Cycloalkyl” refers to a 3 to 8 member all-carbon monocyclic ring, an all-carbon 5-member/6-member or 6-member/6-member fused bicyclic ring, or a multicyclic fused ring (a “fused” ring system means that each ring in the system shares an adjacent pair of carbon atoms with each other ring in the system) group wherein one or more of the rings may contain one or more double bonds but none of the rings has a completely conjugated pi-electron system. Examples, without limitation, of cycloalkyl groups are cyclopropane, cyclobutane, cyclopentane, cyclopentene, cyclohexane, cyclohexadiene, adamantane, cycloheptane, cycloheptatriene, and the like.

“Aryl” refers to an all-carbon monocyclic or fused-ring polycyclic groups of 6 to 12 carbon atoms having a completely conjugated pi-electron system. Examples, without limitation, of aryl groups are phenyl, naphthalenyl and anthracenyl. The aryl group may be substituted or unsubstituted. Typical substituents include halo, trihalomethyl, alkyl, hydroxy, alkoxy, aryloxy, mercapto, alkylthio, arylthio, cyano, nitro, carbonyl, thiocarbonyl, C-carboxy, O-carboxy, O-carbamyl, N-carbamyl, O-thiocarbamyl, N-thiocarbamyl, C-amido, N-amido, sulfinyl, sulfonyl, amino and -NR^{x}R^{Y}, with R^{x} and R^{Y} as defined above.

“Heteroaryl” refers to a monocyclic or fused ring group of 5 to 12 ring atoms containing one, two, three or four ring heteroatoms selected from N, O, and S, the remaining ring atoms being C, and, in addition, having a completely conjugated pi-electron system. Examples, without limitation, of unsubstituted heteroaryl groups are pyrrole, furan, thiophene, imidazole, oxazole, thiazole, pyrazole, pyridine, pyrazine, pyrimidine, quinoline, isoquinoline, purine, tetrazole, triazine, and carbazole. The heteroaryl group may be substituted or unsubstituted. Typical substituents include alkyl, cycloalkyl, halo, trihalomethyl, hydroxy, alkoxy, aryloxy, mercapto, alkylthio, arylthio, cyano, nitro, carbonyl, thiocarbonyl, sulfonamido, C-carboxy, O-carboxy, sulfinyl, sulfonyl, O-carbamyl, N-carbamyl, O-thiocarbamyl, N-thiocarbamyl, C-amido, N-amido, amino and -NR^{x}R^{Y} with R^{x} and R^{Y} as defined above. A pharmaceutically acceptable heteroaryl is one that is sufficiently stable to be attached to a compound of the invention, formulated into a pharmaceutical composition and subsequently administered to a patient in need thereof.

“Heteroalicyclic” or “heterocycle” refers to a monocyclic or fused ring group having in the ring(s) of 3 to 12 ring atoms, in which one or two ring atoms are heteroatoms selected from N, O, and S(O)ₜ, (where t is 0, 1 or 2), the remaining ring atoms being C. The rings may also have one or more double bonds. However, the rings do not have a completely conjugated π-electron system. Additionally, one or more of the ring atoms could be substituted by an oxo group or C₁-C₆ alkyl group. Examples of suitable saturated heteroalicyclic groups include, but are not limited to: tetrahydrofuran, tetrahydrothiophene, pyrrolidine, piperidine, morpholine, piperazine, and 4-methylpiperazine.

“Halogen” means fluorine, chlorine, bromine, and iodine. “Halo” means fluoro, chloro, bromo, and iodo, preferably fluorine or chlorine.

The invention also includes isotopically-labeled compounds of the invention, wherein one or more atoms is replaced by an atom having the same atomic number, but an atomic mass or mass number different from the atomic mass or mass number usually found in nature. Examples of isotopes suitable for inclusion in the compounds of the invention include isotopes of hydrogen, such as deuterium and carbon such as ¹³C. Certain isotopically-labeled compounds of the invention, for example, those incorporating a radioactive isotope, are useful in drug and/or substrate tissue distribution studies. Substitution with heavier isotopes such as deuterium may afford certain therapeutic advantages resulting from greater metabolic stability, for example, increased in vivo half-life or reduced dosage requirements, and hence may be preferred in some circumstances. Isotopically-labeled compounds of the invention can generally be prepared by conventional techniques known to those skilled in the art or by processes analogous to those described herein, using an appropriate isotopically-labeled reagent in place of the non-labeled reagent otherwise employed.

The term “comprising” is meant to be open-ended, including the indicated component(s), but not excluding other elements.

The term “pharmaceutically acceptable” when used with reference to a compound of Formula I or II is intended to refer to a form of the compound that is safe for administration to a subject. For example, a free base, a salt form, a solvate, a hydrate, a prodrug or derivative form of a compound of Formula I or II, which has been approved for mammalian use, via oral ingestion or any other route of administration, by a governing authority or regulatory agency, such as the Food and Drug Administration (FDA) of the United States, is pharmaceutically acceptable.

Included in the compounds of Formula I or II are the pharmaceutically acceptable salt forms of the free-base compounds. The term “pharmaceutically-acceptable salts” embraces salts, commonly used to form alkali metal salts and to form addition salts of free acids or free bases, which have been approved by a regulatory agency. Salts are formed from ionic associations, charge-charge interactions, covalent bonding, complexation, coordination, etc. The nature of the salt is not critical, provided that it is pharmaceutically acceptable.

In some embodiments, the compound(s) of Formula I or II are used to treat a subject by administering the compound(s) as a pharmaceutical composition. To this end, the compound(s), in one embodiment, are combined with one or more pharmaceutically acceptable excipients, including carriers, diluents or adjuvants, to form a suitable composition, which is described in more detail herein.

The term “excipient”, as used herein, denotes any pharmaceutically acceptable additive, carrier, adjuvant, or other suitable ingredient, other than the active pharmaceutical ingredient (API), which is typically included for formulation and/or administration purposes. “Diluent” and “adjuvant” are defined hereinafter.

The terms “treat”, “treating,” “treatment,” and “therapy” as used herein refer to therapy, including without limitation, curative therapy, prophylactic therapy, and preventative therapy. Prophylactic treatment generally constitutes either preventing the onset of disorders altogether or delaying the onset of a pre-clinically evident stage of disorders in individuals.

The phrase “effective amount” is intended to quantify the amount of each agent, which will achieve the goal of improvement in disorder severity and the frequency of incidence over treatment of each agent by itself, while avoiding adverse side effects typically associated with alternative therapies. The effective amount, in one embodiment, is administered in a single dosage form or in multiple dosage forms.

The protection of functional groups by protecting groups, the protecting groups themselves, and their removal reactions (commonly referred to as “deprotection”) are described, for example, in standard reference works, such as J. F. W. McOmie, Protective Groups in Organic Chemistry, Plenum Press, London and New York (1973), T. W. Greene and P. G. M. Wuts, Protective Groups in Organic Synthesis, Wiley, 3^{rd} edition, John Wiley and Sons (1999), E. Gross and J. Meienhofer, The Peptides, Volume 3, Academic Press, London and New York (1981).

The invention further encompasses “intermediate” compounds, including structures produced from the synthetic procedures described, whether isolated or not, prior to obtaining the finally desired compound. Structures resulting from carrying out steps from a transient starting material, structures resulting from divergence from the described method(s) at any stage, and structures forming starting materials under the reaction conditions are all “intermediates” included in the invention. Further, structures produced by using starting materials in the form of a reactive derivative or salt, or produced by a compound obtainable by means of the process according to the invention and structures resulting from processing the compounds of the invention in situ are also within the scope of the invention.

Starting materials of the invention, are either known, commercially available, or can be synthesized in analogy to or according to methods that are known in the art. Many starting materials may be prepared according to known processes and, in particular, can be prepared using processes described in the examples. In synthesizing starting materials, functional groups in some cases are protected with suitable protecting groups when necessary. Protecting groups, their introduction and removal are described above.

The compounds of this invention in some embodiments also are represented in multiple tautomeric forms. The invention expressly includes all tautomeric forms of the compounds described herein.

The compounds in one embodiment also occur in cis- or trans- or E- or Z- double bond isomeric forms. All such isomeric forms of such compounds are expressly included in the present invention.

### Indication

Provided is a method for inhibiting a mutant IDH1 or IDH2 activity comprising contacting a subject in need thereof a compound described in anyone of the embodiments herein, or a pharmaceutically acceptable salt thereof. In one embodiment, the cancer to be treated is characterized by a mutant allele of IDH1 or IDH2 wherein the IDH1 or IDH2 mutation result in a new ability of the enzyme to catalyze the NAPH-dependent reduction of a-ketoglutarate to R(−)-2-hydroxyglutarate in a subject. In one aspect of this embodiment, the mutant IDH1 has an R132X mutation. In one aspect of this embodiment, the R132X mutation is selected from R132H, R132C, R132L, R132V, R132S and R132G. In another aspect, the R132X mutation is R132H or R132C. In yet another aspect, the R132X mutation is R132H.

In one aspect of this embodiment, the efficacy of cancer treatment is monitored by measuring the levels of 2HG in the subject. Typically levels of 2HG are measured prior to treatment, wherein an elevated level is indicative of the use of the compound described herein. Once the elevated levels are established, the level of 2HG is determined during the course of and/or following termination of treatment to establish efficacy. In certain embodiments, the level of 2HG is only determined during the course of and/or following termination of treatment. A reduction of 2HG levels during the course of treatment and following treatment is indicative of efficacy. Similarly, a determination that 2HG levels are not elevated during the course of or following treatment is also indicative of efficacy. Typically, the these 2HG measurements will be utilized together with other well-known determinations of efficacy of cancer treatment, such as reduction in number and size of tumors and/or other cancer-associated lesions, improvement in the general health of the subject, and alterations in other biomarkers that are associated with cancer treatment efficacy.

Provided is a method for inhibiting a mutant IDH2 activity comprising contacting a subject in need thereof a compound described in anyone of the embodiments herein, or a pharmaceutically acceptable salt thereof. In one embodiment, the cancer to be treated is characterized by a mutant allele of IDH2 wherein the IDH2 mutation result in a new ability of cancer treatment efficacy. the enzyme to catalyze the NAPH-dependent reduction of α-ketoglutarate to R(−)-2-hydroxyglutarate in a subject. In one aspect of this embodiment, the mutant IDH2 has an R140X mutation. In another aspect of this embodiment, the R140X mutation is a R140Q mutation. In another aspect of this embodiment, the R140X mutation is a R140W mutation.

In another aspect of this embodiment, the R140X mutation is a R140L mutation. In another aspect of this embodiment, the mutant IDH2 has an R172X mutation. In another aspect of this embodiment, the R172X mutation is a R172K mutation. In another aspect of this embodiment, the R172X mutation is a R172G mutation. Also provided are methods of treating a cancer characterized by the presence of a mutant allele of IDH2 comprising the step of administering to subject in need thereof (a) a compound described in anyone of the embodiments herein, or a pharmaceutically acceptable salt thereof, or (b) a pharmaceutical composition comprising (a) and a pharmaceutically acceptable carrier.

In one embodiment the cancer is a tumor wherein at least 30, 40, 50, 60, 70, 80 or 90% of the tumor cells carry an IDH2 mutation, and in particular an IDH2 R140Q, R140W, or R140L and/or R172K or R172G mutation, at the time of diagnosis or treatment. In another embodiment, one aspect of the invention provides a method of treating a cancer selected from_glioblastoma (glioma), myelodysplastic syndrome (MDS), myeloproliferative neoplasm (MPN), acute myelogenous leukemia (AML), sarcoma, melanoma, non-small cell lung cancer, chondrosarcoma, cholangiocarcinomas or angioimmunoblastic lymphoma in a patient by administering to the patient a compound described herein in an amount effective to treat the cancer. In a more specific embodiment the cancer to be treated is glioma, myelodysplastic syndrome (MDS), myeloproliferative neoplasm (MPN), acute myelogenous leukemia (AML), melanoma, chondrosarcoma, or angioimmunoblastic non-Hodgkin's lymphoma (NHL).

In one embodiment, prior to and/or after treatment with a compound described in anyone of the embodiments described herein, the method further comprises the step of evaluating the IDH2 genotype of the cancer. This may be achieved by ordinary methods in the art, such as DNA sequencing, immuno analysis, and/or evaluation of the presence, distribution or level of 2HG.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

The compounds of the invention may be administered orally. Oral administration may involve swallowing, so that the compound enters the gastrointestinal tract, or buccal or sublingual administration may be employed by which the compound enters the blood stream directly from the mouth. Formulations suitable for oral administration include solid formulations such as tablets, capsules containing particulates, liquids, or powders, lozenges (including liquid-filled), chews, multi- and nanoparticulates, gels, solid solution, liposome, films (including muco-adhesive), ovules, sprays and liquid formulations.

Liquid formulations include suspensions, solutions, syrups and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001), the disclosure of which is incorporated herein by reference in its entirety.

### Combinations

While the compounds of the invention can be dosed or administered as the sole active pharmaceutical agent, they can also be used in combination with one or more compounds of the invention or in conjunction with other agents. When administered as a combination, the therapeutic agents can be formulated as separate compositions that are administered simultaneously or sequentially at different times, or the therapeutic agents can be given as a single composition.

In some embodiments, methods for treatment of androgen receptor-dependent or androgen receptor-mediated conditions or diseases, such as proliferative disorders, including cancer, comprises administration to a mammal a compound of Formula I or II in combination with at least one additional agent selected, by way of example only, alemtuzumab, arsenic trioxide, asparaginase (pegylated or non-), bevacizumab, cetuximab, platinum-based compounds such as cisplatin, cladribine, daunorubicin/doxorubicin/idarubicin, irinotecan, fludarabine, 5-fluorouracil, gemtuzumab, methotrexate, taxol, temozolomide, thioguanine, or classes of drugs including hormones (an antiestrogen, an antiandrogen, or gonadotropin releasing hormone analogues, interferons such as alpha interferon, nitrogen mustards such as busulfan or melphalan or mechlorethamine, retinoids such as tretinoin, topoisomerase inhibitors such as irinotecan or topotecan, tyrosine kinase inhibitors such as gefinitinib or imatinib, or agents to treat signs or symptoms induced by such therapy including allopurinol, filgrastim, granisetron/ondansetron/palonosetron, dronabinol.

Specifically, the administration of compounds of the present invention in some embodiments are in conjunction with additional therapies known to those skilled in the art in the prevention or treatment of cancer. The foregoing description is merely illustrative of the invention and is not intended to limit the invention to the disclosed compounds, compositions and methods.

### Synthesis of Compounds

The compounds of Formula I or II were synthesized according to the procedures described in the following Schemes to those skilled in the art, wherein the substituents are as defined for Formula I or II above, except where further noted. The synthetic methods described below are merely exemplary, and the compounds of the invention may also be synthesized by alternate routes as appreciated by persons of ordinary skill in the art. Compound 1 and Compound 2 are literature known or were prepared readily by following similar literature known procedures. The reaction of Compound 1 and Compound 2 with sodium hydride in solvent such as THF can lead to the synthesis of Compound 3. The methoxy groups of Compound 3 are hydrolyzed to give compound 4, then the POCl₃ reaction to afford Compound 5. The chloride in the 4 position of Compound 5 is replaced by Compound 6 to give Compound 7. The palladium catalyzed coupling reaction of compounds 7 and 8 to give the product Compound 9. (Scheme 1).

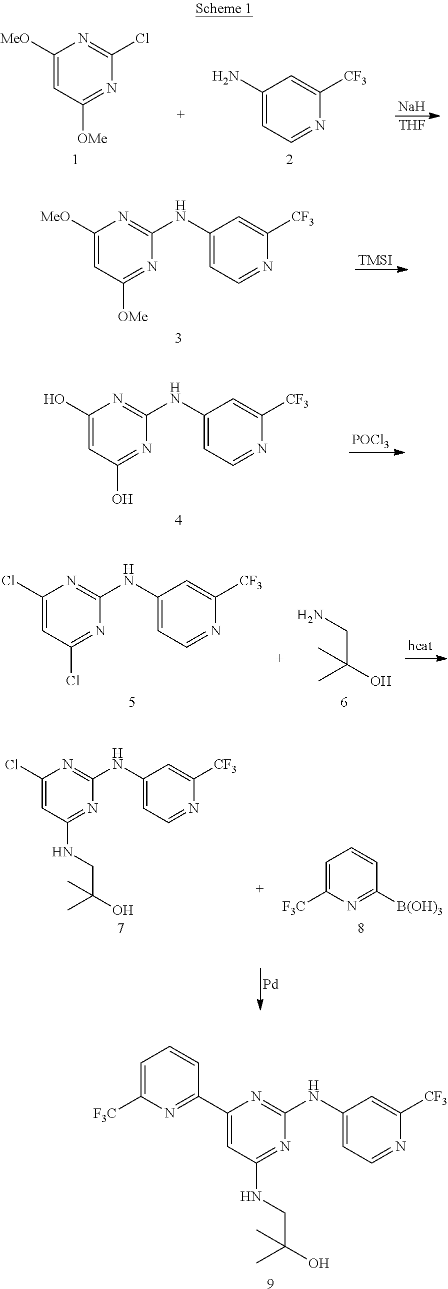

The preparation of Compound 12 is described in Scheme 2. It is prepared by the coupling reaction between two literature known Compounds 10 and 11

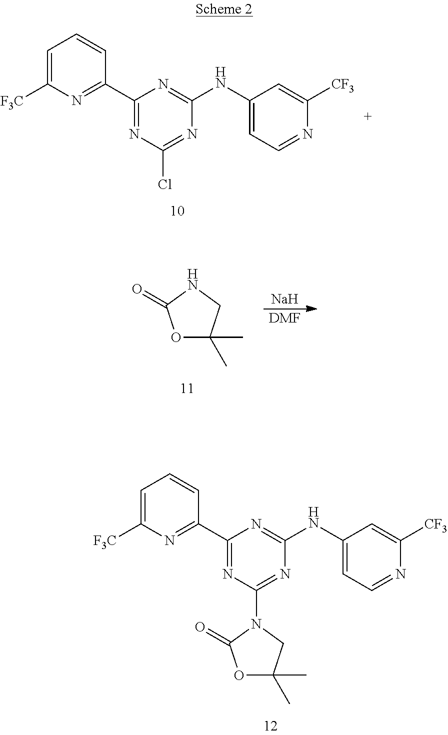

An alternative preparation of Compound 12 is by intramolecular cyclization reaction described in Scheme 3.

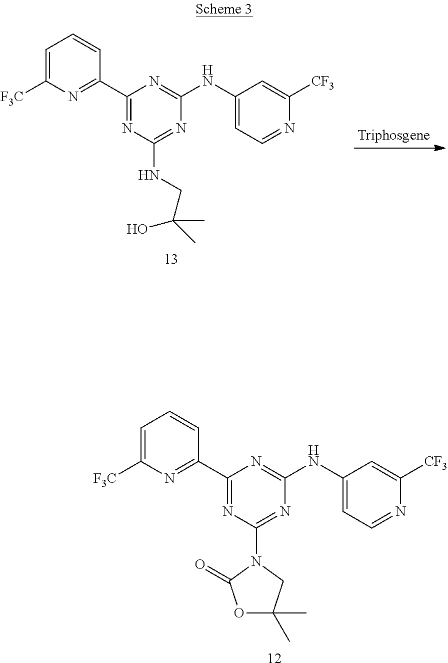

The preparation of Compound 15 and 17 can be followed the synthesis described in Scheme 4. Compound 10 reacts with Compound 14 or 16 with base to give the Compound 15 or 17.

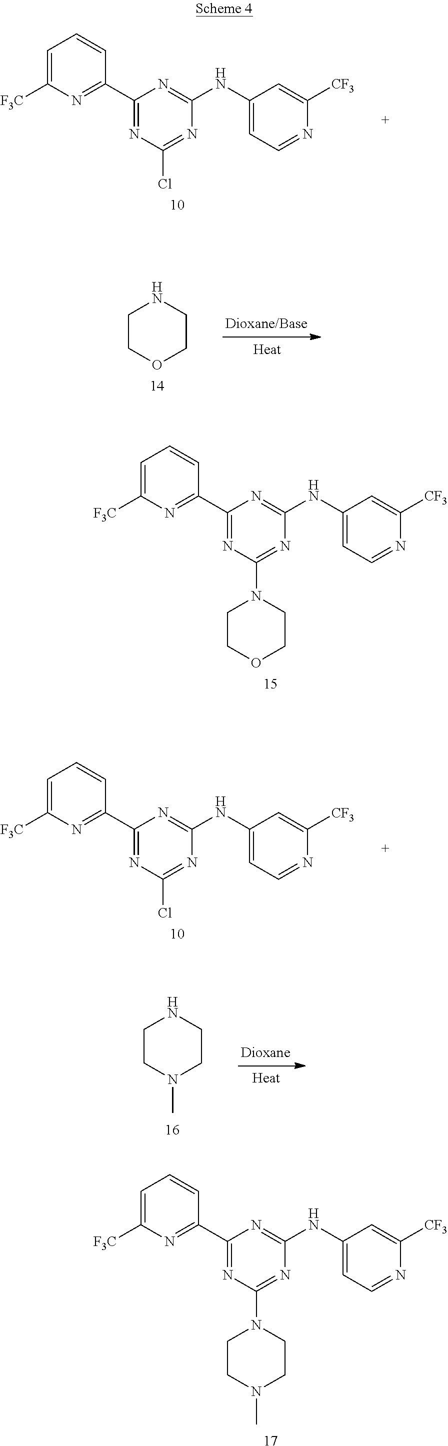

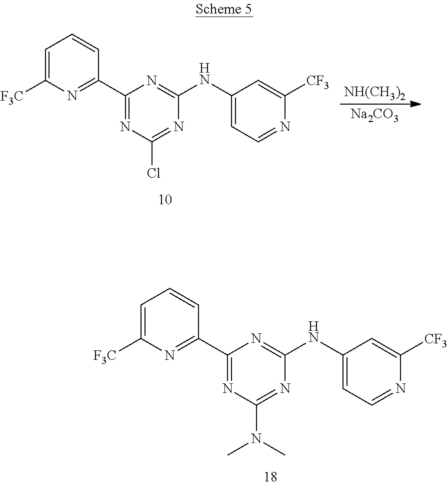

### Description of Embodiments

These detailed descriptions are presented for illustrative purposes only and are not intended as a restriction on the scope of the invention.

### Proton NMR Spectra

Unless otherwise indicated, all ¹H NMR spectra were run on a Varian series Mercury 300, 400 MHz instrument or a Bruker series 400MHz instrument. Where so characterized, all observed protons are reported as parts-per-million (ppm) downfield from tetramethylsilane (TMS) or other internal reference in the appropriate solvent indicated.

### Abbreviation

DCM means dichloromethane.

RT means room temperature.

EA means ethyl acetate.

### EXAMPLE 1

### Preparation of 5,5-dimethyl-3-[4-[6-(trifluoromethyl)-2-pyridyl]-6-[[2-(trifluoromethyl)-4-pyridyl]amino]-1,3,5-triazin-2-yl]oxazolidin-2-one (Compound 12)

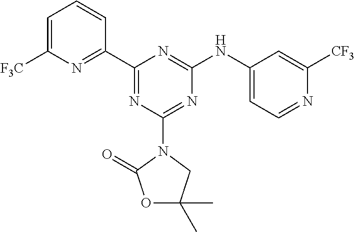 [SMILES: CC1(C)CN(c2nc(Nc3ccnc(C(F)(F)F)c3)nc(-c3cccc(C(F)(F)F)n3)n2)C(=O)O1]

To a solution of 2-methyl-1-[ [4-[6-(trifluoromethyl)-2-pyridyl]-6-[ [2-(trifluoromethyl)-4-pyridyl]amino]-1,3,5-triazin-2-yl]amino]propan-2-ol (100 mg, 1.0 equiv) is added triphosgene in DCM at −78° C., followed by dropwise addition of 2,6-lutidine. The reaction is warmed to room temperature and heated at 35° C. for 5 hours. All the solvent is removed under reduce pressure, and purification by flash chromatograph to give desire product.

### EXAMPLE 2

### Preparation of 4-(4-methylpiperazin-1-yl)-6-(6-(trifluoromethyl)pyridin-2-yl)-N-(2-(trifluoromethyl)pyridin-4-yl)-1,3,5-triazin-2-amine (Compound 17)

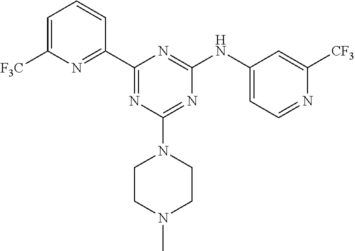 [SMILES: CN1CCN(c2nc(Nc3ccnc(C(F)(F)F)c3)nc(-c3cccc(C(F)(F)F)n3)n2)CC1]

To a solution of 2,4-dichloro-6-(6- (trifluoromethyl)pyridin-2-yl)-1,3,5-triazine (200 mg, 0.678 mmol, 1.00 equiv) and 2-(trifluoromethyl)pyridine-4-amine (120 mg, 0.741 mmol, 1.1 eq) in THF (4 mL) were added Na₂CO₃ (105 mg, 1.00 mmol, 1.50 eq). The solution is stirred for at rt for overnight, then H₂O (30 ml) and EA (30 ml) were added. The organic layer was washed with 1N HCl, and saturated NaCl solution, dried over with anhydrous Na₂SO₄, and concentrated under reduced pressure to afford a yellow oil. The oil was dissolved in THF (4 ml), then 1-methylpiperazine (80 mg, 0.800 mmol, 1.20 eq) and Na₂CO₃ (105 mg, 1.00mmol, 1.50 eq) are added and stirred at rt for overnight. 6 ml H₂O and 6 ml EA were added into. The organic layer is concentrated under reduced pressure. Purification by flash column chromatography on silica gel to give 4-(4-methylpiperazin-1-yl)-6-(6-(trifluoromethyl) pyridin-2-yl)-N-(2-(trifluoromethyl)pyridin-4-yl)-1,3,5-triazin-2-amine (30 mg). ¹H-NMR(DMSO-d⁶, 400 Hz): δ2.25(s, 3H), δ2.44(m, 4H), δ3.87(m, 2H), δ3.97(m, 2H), 7.90(dd, J=5.6 Hz, J=1.6 Hz, 1H), 8.10(dd, J=8.0 Hz, J=0.8 Hz, 1H), 8.29(t, J=8.0 Hz, 1H), 8.57(d, J=5.6 Hz, 1H), 8.67(d, J=8.0 Hz, 1H), 610.74(s, 1H). MS m/z: 485 [M+1].

### EXAMPLE 3

### Preparation of N²,N²-dimethyl-6-(6-(trifluoromethyl)pyridin-2-yl)-N⁴-(2-(trifluorome thyl)pyridin-4-yl)-1,3,5-triazine-2,4-diamine (Compound 18)

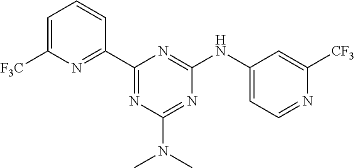 [SMILES: CN(C)c1nc(Nc2ccnc(C(F)(F)F)c2)nc(-c2cccc(C(F)(F)F)n2)n1]

The 2,4-dichloro-6-(6- (trifluoromethyl)pyridin-2-yl)-1,3,5-triazine (300 mg, 1.017mmol, 1 eq) and 2-(trifluoromethyl)pyridine-4-amine (180 mg, 1.111mmol, 1.1 eq) are added to THF (6 mL) then Na₂CO₃ (160 mg, 1.524 mmol, 1.5 eq). The solution is stirred for overnight at rt, then 20 ml H2O and 20 ml EA are added into. The organic layer is washed with 1N HCl, and saturate NaCl solution, dried with anhydrous Na₂SO₄, concentrated under reduced pressure to get yellow oil. The oil is dissolved in 6 ml THF, dimethylamine hydrochloride (300 mg, 3.703 mmol, 3.64 eq) and Na₂CO₃ (220 mg, 2.00 mmol, 2.1 eq) are added into and stirred for overnight at rt, then 20 ml H₂O and 20 ml EA were added into. The organic layer is washed with saturate NaCl solution, dried with anhydrous Na₂SO₄, concentrated under reduced pressure to get crude product and purified by column chromatography on silica gel to give N²,N²-dimethyl-6-(6-(trifluoromethyl) pyridine-2-yl)-N⁴-(2-(trifluoromethyl)pyridin-4-yl)-1,3,5-triazine-2,4-diamine (50 mg). Yield (11.5%). ¹H-NMR(DMSO-d⁶, 400 Hz): δ3.25(s, 3H), 7.93(d, J=5.2 Hz, 1H), 8.10(d, J=7.6 Hz, 1H), 8.30(t, J=7.6 Hz, 1H), 8.56(d, J=5.2 Hz, 1H), 8.67(d, J=8.0 Hz, 1H), 8.70(m, 1H), δ10.72(s, 1H). MS m/z: 485 [M+1].

### EXAMPLE 4

### Preparation of Compound 19 (AG-221)

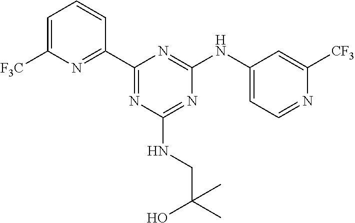 [SMILES: CC(C)(O)CNc1nc(Nc2ccnc(C(F)(F)F)c2)nc(-c2cccc(C(F)(F)F)n2)n1]

Compound 19 was prepared according to Scheme 4 following by the literature known procedure. BIOLOGICAL ASSAYS:

IDH2 Enzymatic Assay.

Enzymatic Assay. Compounds were assayed for IDH2 R140Q inhibitory activity through a cofactor depletion assay. Compounds were preincubated with enzyme, then the reaction was started by the addition of NADPH and a-KG, and allowed to proceed for 60 minutes under conditions previously demonstrated to be linear with respect for time for consumption of both cofactor and substrate. The reaction was terminated by the addition of a second enzyme, diaphorase, and a corresponding substrate, resazurin. Diaphorase reduces resazurin to the highly fluorescent resorufin with the concomitant oxidation of NADPH to NADP, both halting the IDH2 reaction by depleting the available cofactor pool and facilitating quantitation of the amount of cofactor remaining after a specific time period through quantitative production of an easily detected fluorophore. Specifically, into each of 12 wells of a 384-well plate, 1 μl of compound dilution series was placed, followed by the addition of 40 μl of buffer (50 mM potassium phosphate, pH 7.5; 150 mM NaCI; 10 mM MgCl, 10% glycerol, 0.05% bovine serum albumin, 2 mM beta-mercaptoethanol) containing 1.25 μg/ml IDH2 R140Q. The compound was then incubated for one hour at room temperature with the enzyme; before starting the IDH2 reaction with the addition of 10 μl of substrate mix containing 50 μM NADPH and 6.3 mM a-KG in the buffer described above. After a further one hour of incubation at room temperature, the reaction was halted and the remaining NADPH measured through conversion of resazurin to resorufin by the addition of 25 μl Stop Mix (36 μg/ml diaphorase enzyme and 60 resazurin; in buffer). After one minute of incubation the plate was read on a plate reader at Ex544/Em590.

TABLE A
Enzyme Inhibition

| Enzyme | Compound 19 | Compound17 | Compound18 |
|---|---|---|---|
| IDH2 | 401 nM | 262 nM | 12.2 nM |

As stated hereinbefore, the compounds defined in the present invention possess anti-proliferation activity. These properties may be assesses, for example, using one or more of the procedures set out below:
An in vitro assay which determines the ability of a test compound to inhibit kinases. Conducts the kinase assay with the following condition and procedure:
Reagent: Base Reaction Buffer; 20 mM Hepes (pH 7.5), 10 mM MgCl₂, 1 mM EGTA, 0.02% Brij35, 0.02 mg/ml BSA, 0.1 mM Na₃VO₄, 2 mM DTT, 1% DMSO.

### Reaction Procedure:

1. Prepare indicated substrate in freshly prepared Base Reaction Buffer.
2. Deliver any required cofactors to the substrate solution obtained in step 1.
3. Deliver indicated kinase into the substrate solution prepared in step 2 and gently mix.
4. Deliver compounds in DMSO into the kinase reaction mixture prepared in step 3.
5. Deliver ³³P-ATP (specific activity 0.01 μCi/μl final) into the reaction mixture prepared in step 4 to initiate the reaction.
6. Incubate kinase reaction for 120 min. at room temperature.
7. Reactions are spotted onto P81 ion exchange paper (Whatman # 3698-915).
8. Washing filters extensively in 0.75% Phosphoric acid.
   Compound was tested in single dose duplicate mode at a concentration of 10 μM, reactions were carried out at 10 μM ATP

TABLE B
Kinase Inhibition of Compound17

% Enzyme Activity
(relative to
DMSO controls)

| Data 1 | Data 2 | Kinases |
|---|---|---|
| 79.17 | 78.86 | ALK (L1152 R) |
| 58.23 | 58.12 | AXL (R499C) |
| 69.91 | 68.86 | BRAF (G464V) |
| 79.85 | 79.49 | c-MER (A708S) |

Compound 17 was tested against a panel of kinases and found to be active against kinases ALK (L1152 R), AXL (R499C), BRAF (G464V) and c-MER (A708S).

Compound 17 also has better pharmacokinecitc (PK) and favorable safety profile than Compound 19.

### Solubility Measurement:

Preparation of reference standard solution: 2 mg of Compound 17 or Compound 19 was added individually to 100 mL volumetric flask each. The compound was diluted with acetonitrile to 100 mL.

Preparation of sample solution: 2 mg of Compound 17 or Compound 19 was added individually to 2 mL eppendorf tube (EP), followed by addition of 1 mL of pH 7.0 or 4.0 buffer solution (20 mM). The solution was shook for 2 minutes and left for 30 minutes at 25° C. After standing for 30 minutes, precipitate was formed in the bottom of the EP. The solutions was filtered through 0.2 um membrane filter, and then diluted by 50 times with water.

The standard and sample solutions were injected into the HPLC on a Shim-Pack CLC-ODS C₁₈ column (150mm×6.0mm, 5um) with the same volume. The mobile phase consisted of acetonitrile with 2% trichloromethane−20 mM KH₂PO₄ buffer (pH=7.0) at a flow rate of 1 mL/minutes (40:60). The detection wavelength is at 264 nm. Calculation: solubility of sample=the concentration of standard×Area of sample×50 /Area of standard. Compound 17 had a better solubility to Compound 19 at pH=7.0, but had much higher solubility than Compound 19 at pH=4.0.

TABLE C
Solubility of Compounds of the invention

| | Compound 17 | Compound 19 |
|---|---|---|
| Solubility at pH 4.0 | 123 μg/mL | 1.59 μg/mL |
| Solubility at pH 7.0 | 0.276 μg/mL | 0.109 μg/mL |

1-11. (canceled)

12. A compound according to Formula I: 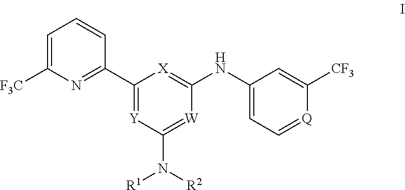
or a pharmaceutically acceptable salt, solvate, stereoisomer, tautomer or prodrug thereof, wherein
R¹ and R² are independently —C(O)OR³, —C(O)R³, —SO₂R³, or C₁-C₆ alkyl, optionally substituted by one or more R⁴, or R¹ and R², together with the nitrogen attached thereto, form a 3-12 membered heteroalicyclic ring;
R³ is C₁-C₆ alkyl, optionally substituted by one or more R⁴;
R⁴is halogen, OH, C₁-C₆ alkoxy, NH(CO)R⁵R⁶, or NR⁵R⁶;
R⁵ and R⁶ are independently hydrogen or C₁-C₆ alkyl; and
X, Y, W and Q are independently N or CH.

13. The compound of claim 12, wherein X, Y, W and Q are N.

14. The compound of claim 12, wherein X, W and Q are N, and Y is CH.

15. A pharmaceutical composition comprising a compound of claim 12 or a pharmaceutically acceptable salt thereof and a pharmaceutically acceptable carrier.

16. A method for treating a disorder in an individual, comprising: administering to the individual a therapeutically effective amount of a pharmaceutical composition, the pharmaceutical composition comprising a compound of claim 12 or a pharmaceutical acceptable salt thereof and a pharmaceutically acceptable carrier, wherein the disorder is a cancer characterized by an IDH2 mutation, and wherein the IDH2 mutation enables the corresponding isocitrate dehydrogenase to catalyze an NADPH-dependent reduction of a-ketoglutarate to R(−)-2-hydroxyglutarate.

17. The method of claim 16, wherein the IDH2 mutation is an IDH2 R140Q or R172K mutation.

18. The method of claim 16, wherein the cancer is neoplasia or acute lymphocytic leukemia.

19. The method of claim 16, further comprising a step of co-administering to the individual with one or more anti-cancer agents, wherein the cancer is neoplasia.

20. A compound according to Formula II: 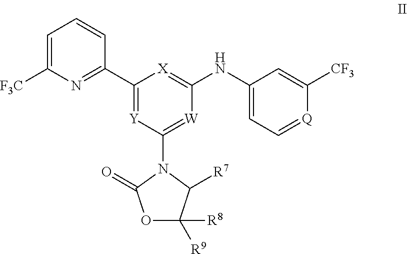
or a pharmaceutically acceptable salt, solvate, stereoisomer, tautomer or prodrug thereof,
wherein
R⁷, R⁸ and R⁹ are independently hydrogen, or C₁-C₆ alkyl, optionally substituted by one or more R⁴;
R⁴ is halogen, OH, C₁-C₆ alkoxy, NH(CO)R⁵R⁶, or NR⁵R⁶;
R⁵ and R⁶ are independently hydrogen or C₁-C₆ alkyl; and
X, Y, W and Q are independently N or CH.

21. The compound of claim 20, wherein X, Y, W and Q are N.

22. The compound of claim 20, wherein X, W and Q are N, and Y is CH.

23. A pharmaceutical composition comprising a compound of claim 20 or a pharmaceutically acceptable salt thereof and a pharmaceutically acceptable carrier.

24. A method for treating a disorder in an individual, comprising: administering to the individual a therapeutically effective amount of a pharmaceutical composition, the pharmaceutical composition comprising a compound of claim 20 or a pharmaceutical acceptable salt thereof and a pharmaceutically acceptable carrier, wherein the disorder is a cancer characterized by an IDH2 mutation, and wherein the IDH2 mutation enables the corresponding isocitrate dehydrogenase to catalyze an NADPH-dependent reduction of a-ketoglutarate to R(−)-2-hydroxyglutarate.

24. The method of claim 24, wherein the IDH2 mutation is an IDH2 R140Q or R172K mutation.

25. The method of claim 24, wherein the cancer is neoplasia or acute lymphocytic leukemia.

26. The method of claim 24, further comprising a step of co-administering to the individual with one or more anti-cancer agents, wherein the cancer is neoplasia.

27. A compound or a pharmaceutically acceptable salt or solvate thereof, wherein the compound is selected from the group consisting of: 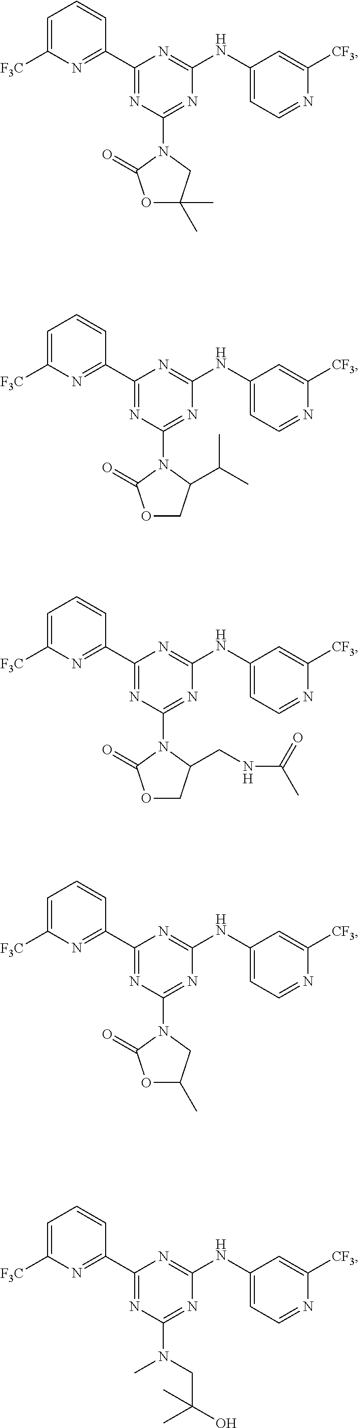 [SMILES: CC1(C)CN(c2nc(Nc3ccnc(C(F)(F)F)c3)nc(-c3cccc(C(F)(F)F)n3)n2)C(=O)O1 ; CC(C)C1COC(=O)N1c1nc(Nc2ccnc(C(F)(F)F)c2)nc(-c2cccc(C(F)(F)F)n2)n1 ; CC(=O)NCC1COC(=O)N1c1nc(Nc2ccnc(C(F)(F)F)c2)nc(-c2cccc(C(F)(F)F)n2)n1 ; CC1CN(c2nc(Nc3ccnc(C(F)(F)F)c3)nc(-c3cccc(C(F)(F)F)n3)n2)C(=O)O1 ; CN(CC(C)(C)O)c1nc(Nc2ccnc(C(F)(F)F)c2)nc(-c2cccc(C(F)(F)F)n2)n1] 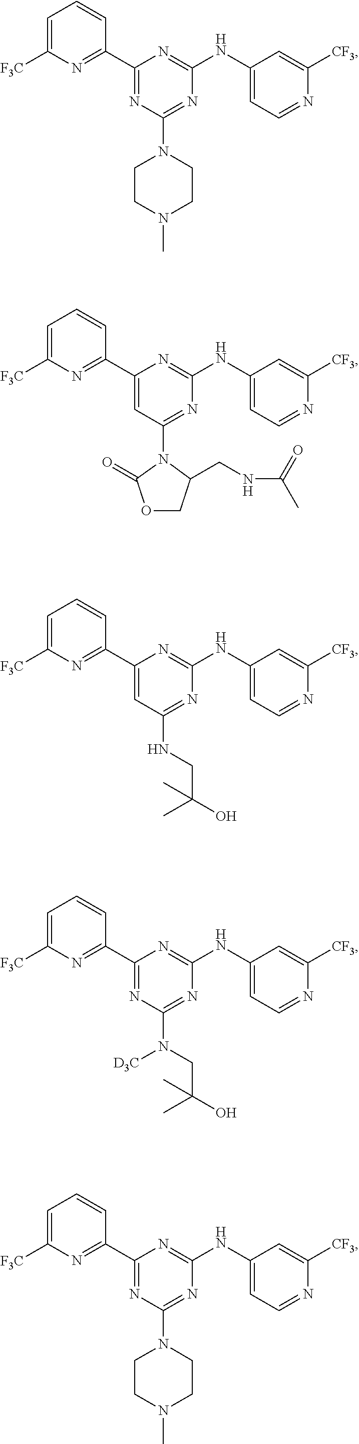 [SMILES: CN1CCN(c2nc(Nc3ccnc(C(F)(F)F)c3)nc(-c3cccc(C(F)(F)F)n3)n2)CC1 ; CC(=O)NCC1COC(=O)N1c1cc(-c2cccc(C(F)(F)F)n2)nc(Nc2ccnc(C(F)(F)F)c2)n1 ; CC(C)(O)CNc1cc(-c2cccc(C(F)(F)F)n2)nc(Nc2ccnc(C(F)(F)F)c2)n1 ; [2H]C([2H])([2H])N(CC(C)(C)O)c1nc(Nc2ccnc(C(F)(F)F)c2)nc(-c2cccc(C(F)(F)F)n2)n1 ; CN1CCN(c2nc(Nc3ccnc(C(F)(F)F)c3)nc(-c3cccc(C(F)(F)F)n3)n2)CC1] 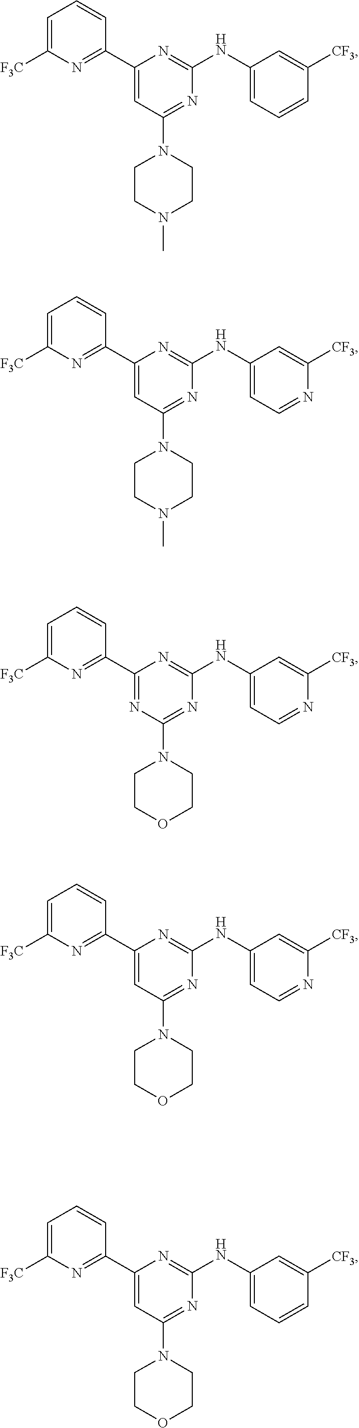 [SMILES: CN1CCN(c2cc(-c3cccc(C(F)(F)F)n3)nc(Nc3cccc(C(F)(F)F)c3)n2)CC1 ; CN1CCN(c2cc(-c3cccc(C(F)(F)F)n3)nc(Nc3ccnc(C(F)(F)F)c3)n2)CC1 ; FC(F)(F)c1cc(Nc2nc(-c3cccc(C(F)(F)F)n3)nc(N3CCOCC3)n2)ccn1 ; FC(F)(F)c1cc(Nc2nc(-c3cccc(C(F)(F)F)n3)cc(N3CCOCC3)n2)ccn1 ; FC(F)(F)c1cccc(Nc2nc(-c3cccc(C(F)(F)F)n3)cc(N3CCOCC3)n2)c1] 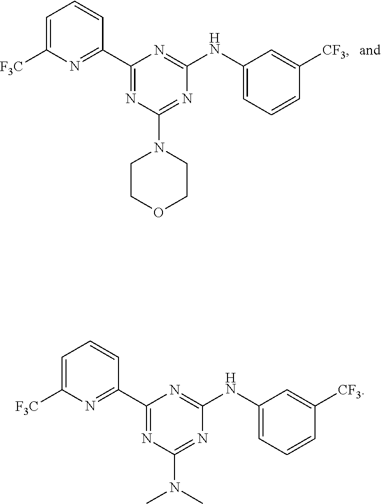 [SMILES: FC(F)(F)c1cccc(Nc2nc(-c3cccc(C(F)(F)F)n3)nc(N3CCOCC3)n2)c1 ; "and" ; CN(C)c1nc(Nc2cccc(C(F)(F)F)c2)nc(-c2cccc(C(F)(F)F)n2)n1]
